(19) United States
(12) Patent Application Publication
(10) Pub. No.: US 2023/0173309 A1
(43) Pub. Date: Jun. 8, 2023

(54) NEGATIVE PRESSURE PROCEDURE MASK

(71) Applicant: UNIVERSITY OF SOUTHERN CALIFORNIA, Los Angeles, CA (US)

(72) Inventors: Brian Applegate, Los Angeles, CA (US); Elisabeth Ference, Los Angeles, CA (US); Wihan Kim, Los Angeles, CA (US); John Oghalai, Los Angeles, CA (US)

(21) Appl. No.: 17/923,769

(22) PCT Filed: Jun. 1, 2021
(86) PCT No.: PCT/US2021/035280

§ 371 Date: Nov. 7, 2022

Related U.S. Application Data
(60) Provisional application No. 63/033,052, filed on Jun. 1, 2020.

(51) Int. Cl. A62B 7/10; A62B 18/00; A62B 18/08; A62B 9/00; G01N 15/06; A41D 13/11

(52) U.S. Cl. CPC A62B 7/10; A62B 18/006; A62B 18/08; A62B 9/006; G01N 15/06; A41D 13/1107; G01N 2015/0693

(57) ABSTRACT

A mask for a patient and a method for reducing exposure of aerosol particles emitted from the patient to other individuals. The mask may have an inner chamber configured to enclose a nose and a mouth of the patient. The mask may further have a negative pressure chamber fluidly connected to the inner chamber. The negative pressure chamber may have an outer opening and a suction opening. The outer opening may be covered by a seal. The seal may be configured to be cut or opened to form a device opening providing access to the nose of the patient. The device opening may receive a medical device and form a seal around the medical device. The suction opening may be configured to couple to a suction device for removing the aerosol particles located within the inner chamber and the negative pressure chamber.

### CROSS REFERENCE TO RELATED APPLICATIONS

This application claims the benefit and priority of U.S. Provisional Application No. 63/033,052, entitled “Negative Pressure Procedure Mask,” filed on Jun. 1, 2020, the contents of which are hereby incorporated by reference in its entirety herein.

### BACKGROUND

### 1. Field

The present disclosure relates to medical face coverings, particularly to a negative pressure procedure mask and a method of using the same.

### 2. Description of the Related Art

Aerosols, such as those entrained with the SARS-CoV-2 virus, are suspected as a primary method of viral transmission. Notably, high concentration of virus particles haven been measured in the upper respiratory system of patients infected with various SARS Coronaviruses. These aerosols are commonly generated and expelled during procedures invasive to the nasopharynx. Consequently, otolaryngologists, or ear, nose, and throat (ENT) physicians, and staff are particularly vulnerable during common clinical procedures, such as nasal endoscopy and laryngoscopy, on infected patients due to their work in the nasal cavity and the nasopharynx. In such clinical procedures, viral droplets may be converted into aerosols, thereby increasing the infectiousness of the virus and the level of personal protective equipment (PPE) required. Further, when the virus is aerosolized, any following patient who enters a medical examination room while the virus is still viable may be infected. For example, a single endoscopic skull base surgery reportedly resulted in fourteen operating room healthcare workers becoming infected with COVID-19.

ENT physicians perform endoscopy procedures in medical examination rooms for surveillance of neoplasms or inflammatory conditions and diagnostic purposes. Patients may be asymptomatic carriers of a virus or may present isolated and mild symptoms, such as anosmia, which may not be recognized as an infection. For example, a recent study has shown that nasal endoscopy, along with sneezing caused by endoscopy, generates particles in the 1-10 micrometer (μm) range. In some known medical practices during the COVID-19 pandemic, all patients with planned clinical procedures had to undergo COVID-19 testing one to three days prior to their procedure date, the physicians and the staff had to wear PPE, including N95 masks, face shields, and goggles, the procedure room included a separate air circulation from the rest of the clinic, and the room was terminally cleaned between each patient. Such protocols are not feasible for routine patient care as they severely limit the capacity of the clinic, are costly, and testing may be limited.

Prior studies have determined that masking a patient may decrease the number of aerosols expelled into the environment with coughing or sneezing. Currently, no patient mask is known to be suitable for and used in these procedures. Thus, a negative pressure procedure mask to reduce exposure of aerosol particles emitted from the patient to other individuals is needed.

### SUMMARY

Described herein are a negative pressure procedure mask and a method thereof to reduce exposure of aerosol particles emitted from a patient to other individuals. The negative relative pressure inside the mask may create a positive flow to house suction. This may mitigate any aerosols created during a procedure or medical examination from entering the exam room. Aerosol detectors integrated with the mask may inform a physician and medical staff when an aerosol has been produced and if it has leaked from the mask. Ports may allow medical tools, such as endoscopic tools, to reach into the mask while creating a seal to maintain the negative pressure.

A mask for a patient for reducing exposure of aerosol particles emitted from the patient to other individuals may have an inner chamber configured to enclose a nose and a mouth of the patient. The mask may further have a negative pressure chamber fluidly connected to the inner chamber and having an outer opening and a suction opening. The outer opening may be covered by a seal. The suction opening may be configured to couple to a suction device for removing the aerosol particles located withing the inner chamber and the negative pressure chamber. The seal may be cut or opened to form a device opening providing access to the nose of the patient. The device opening may receive a medical device and form a seal around the medical device. The medical device may be an endoscope.

The mask may further have an outer wall. The outer wall may define a posterior opening for surrounding the nose and the mouth of the patient. The outer wall may further define the suction opening of the negative pressure chamber. The outer wall may further define the outer opening of the negative pressure chamber. The outer wall may further define a filter opening configured to receive and house a filter for filtering air moving between the inner chamber and outside the mask. The mask may further have an inner wall defining the negative pressure chamber.

The mask may further have a first aerosol sensor located at or near the suction opening. The first aerosol sensor may be configured to detect the aerosol particles being removed by the suction device. The mask may further have a second aerosol sensor located outside of the mask. The second aerosol sensor may be configured to detect the aerosol particles escaping the negative pressure chamber. The first aerosol sensor and the second aerosol sensor may use light scattering to detect the aerosol particles.

A mask for a patient to be worn during a medical examination to mitigate exposure of aerosol particles emitted from the patient into an examination room may have a negative pressure chamber. The negative pressure chamber may have an outer opening and a suction opening. The suction opening may be configured to couple to a suction device for removing the aerosol particles located within the negative pressure chamber. The mask may further have a seal covering the outer opening. The seal may have a device opening providing access to a nose of the patient. The device opening may receive a medical device and form a seal around the medical device. The device opening may be cut with a sharp instrument or opened prior to receiving the medical device. The medical device may be an endoscope. The mask may further have an inner chamber fluidly connected to the negative pressure chamber and configured to enclose the nose and a mouth of the patient.

The mask may further have an outer wall. The outer wall may define a posterior opening for surrounding the nose and the mouth of the patient. The outer wall may further define the suction opening of the negative pressure chamber. The outer wall may further define the outer opening of the negative pressure chamber. The outer wall may further define a filter opening configured to receive and house a filter for filtering air moving between the inner chamber and outside the mask. The mask may further have an inner wall defining the negative pressure chamber.

The mask may further have a first aerosol sensor located at or near the suction opening. The first aerosol sensor may be configured to detect the aerosol particles being removed by the suction device. The mask may further have a second aerosol sensor located outside of the mask. The second aerosol sensor may be configured to detect the aerosol particles escaping the negative pressure chamber. The first aerosol sensor and the second aerosol sensor may use light scattering to detect the aerosol particles.

A method for reducing exposure of aerosol particles emitted from a patient to other individuals may include covering at least a nose and a mouth of the patient using a mask. The mask may have an inner chamber configured to enclose the nose and the mouth of the patient. The mask may further have a negative pressure chamber fluidly connected to the inner chamber and having an outer opening and a suction opening. The outer opening may be covered by a seal. The method may further include connecting a suction device to the suction port. The suction device may be configured to remove the aerosol particles located within the negative pressure chamber and the inner chamber. The method may further include cutting or opening the seal to form a device opening providing access to the nose of the patient. The method may further include inserting a medical device into the device opening. The device opening may form a seal around the medical device.

The method may further include detecting whether the aerosol particles are escaping the mask using a first aerosol sensor located at the suction opening. The first aerosol sensor may be configured to detect the aerosol particles being removed by the suction device. The method may further include detecting whether the aerosol particles are escaping the mask using a second aerosol sensor located outside of the mask. The second aerosol mask may be configured to detect the aerosol particles escaping the negative pressure chamber.

### BRIEF DESCRIPTION OF THE DRAWINGS

Other systems, methods, features, and advantages of the present invention will be or will become apparent to one of ordinary skill in the art upon examination of the following figures and detailed description. It is intended that all such additional systems, methods, features, and advantages be included within this description, be within the scope of the present invention, and be protected by the accompanying claims. Component parts shown in the drawings are not necessarily to scale and may be exaggerated to better illustrate the important features of the present invention.

FIG. 1 illustrates a front view of a patient wearing a negative pressure procedure mask according to an aspect of the present disclosure;

FIG. 2 illustrates a side view of the patient wearing the negative pressure procedure mask according to an aspect of the present disclosure;

FIG. 3 illustrates a cross section view of the patient wearing the negative pressure procedure mask according to an aspect of the present disclosure;

FIG. 4 illustrates an aerosol sensor of the negative pressure procedure mask using light scattering to detect aerosol particles according to an aspect of the present disclosure; and

FIG. 5 is a flowchart illustrating a method for reducing exposure of aerosol particles emitted from a patient to other individuals according to an aspect of the present disclosure.

### DETAILED DESCRIPTION

The negative pressure procedure mask and the method described herein may reduce exposure of aerosol particles emitted from a patient to other individuals. The mask may have a plurality of chambers. An inner chamber may enclose a nose and a mouth of the patient. A negative pressure chamber may be fluidly connected to the inner chamber and coupled to a suction device for removing aerosol particles located within the negative pressure chamber and the inner chamber. The negative pressure within the negative pressure chamber may advantageously prevent any aerosol from exiting the mask. The negative pressure chamber may have a seal acting as an outer wall to the negative pressure chamber. The seal may have an opening or be cut to form an opening to provide access to the nose of the patient. The opening may receive a medical device and form a seal around the medical device. The mask may have a filter opening for filtering air moving between the outside of the mask and the inner chamber. The mask may have a plurality of aerosol sensors. A first aerosol sensor may be located near an outlet to the suction device to detect the aerosol particles removed by the suction device. A second aerosol sensor may be located outside of the mask to detect the aerosol particles escaping the negative pressure chamber.

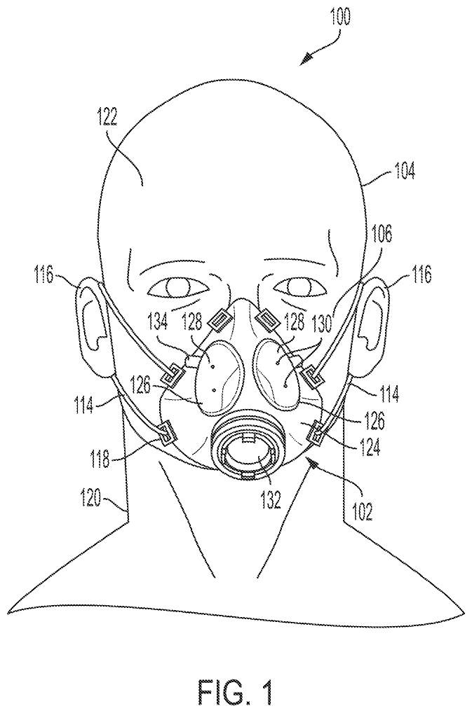

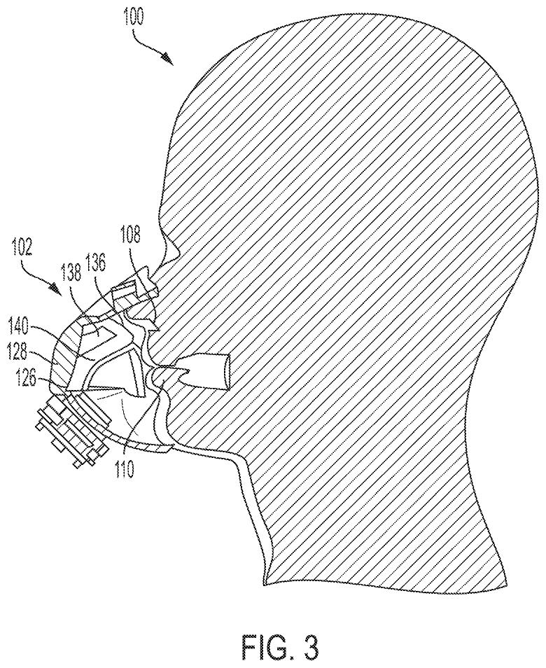

FIG. 1 illustrates a front view of a patient 100 wearing a negative pressure procedure mask 102 according to an aspect of the present disclosure. The mask 102 may be worn around a head 104 of the patient 100 to cover a portion of a face 106 of the patient 100. Particularly, the mask 102 may cover a nose 108 (see FIG. 3) and a mouth 110 (see FIG. 3) of the patient 100. The mask 102 may be attached to the patient 100 by straps or loops 114 that each go over an ear 116 of the patient 100. The straps or loops 114 may be attached to the mask 102 using buckles 118. The buckles 118 may be disposed along the edges of the mask 102. In some embodiments, there may be buckles 118 disposed at a variety of locations on the mask 102 to suit various loop attachments and configurations as shown in FIG. 1. As such, not all buckles 118 may be used at once. In some embodiments, holes, hooks, and other conventional attachment techniques may be used in place of or in addition to the buckles 118 to secure the straps or loops 114 to the mask 102. The straps or loops 114 may be elastic bands. Other ways to attach the mask 102 to the patient 100 are contemplated, such as arms that extend behind the ears 116 and securements around the neck 120 or the forehead 122 of the patient 100.

The mask 102 may have an outer wall 124. The outer wall 124 may define one or more outer openings 126. Two outer openings 126 are shown in FIG. 1 by example. The two outer openings 126 may be located or positioned on left and right sides of the mask 102. The outer openings 126 may each be covered by a seal 128. The seals 128 may be made from silicone. In some embodiments, the seals 128 may be made from another type of polymer that is flexible enough to allow easy movement of a medical device, but hard enough to avoid collapsing due to negative pressure. For example, blind grommets may be used as the seals 128. In some embodiments, the seals 128 may have one or more device openings 130. The device openings 130 may provide access to the nose 108 of the patient 100. The device openings 130 may receive a medical device and form a seal around the medical device. For example, the medical device may be an endoscope or a laryngoscope. The device openings 130 may be shaped and sized to accommodate a wide range of patient anatomies. In some embodiments, the seals 128 may be cut with a scalpel or another sharp medical-grade tool to form the device openings 130. In some embodiments, the seals 128 may be punctured using a pin or needle to form the device openings 130.

The outer wall 124 may further define one or more filter openings 132. One filter opening 132 is shown in FIG. 1 by example. The filter opening 132 may receive and house a filter for filtering the air moving between the two sides of the outer wall 124, or, said differently, the inside of the mask 102 and the outside of the mask 102. The filter opening 132 may be located or positioned at or near a center of the mask 102 below the outer openings 126. The filter may be a high-efficiency particulate air (HEPA) filter that can filter more than 99.97% of 0.3 μm particles or another conventional filter.

The outer wall 124 may further define a suction opening 134. The suction opening 134 may be coupled to a remote or external suction device for removing aerosol particles located inside the mask 102. Suction devices may be commonly available in examination rooms. The suction device may provide air flow through the mask 102 and a slight relative negative pressure to prevent aerosols from exiting the mask 102.

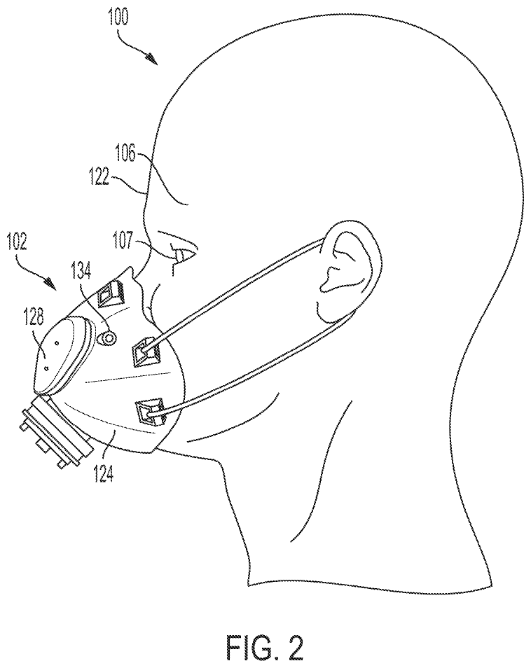

FIG. 2 illustrates a side view of the patient 100 wearing the negative pressure procedure mask 102 according to an aspect of the present disclosure. The suction opening 134 may be on each side of the mask 102. An unused suction opening 134 may be sealed with a cover. The cover may be the same material as the seals 128 or another type of polymer. The mask 102 may conform to the profile of the face 106 of the patient 100. The mask 102 may have a rounded outer wall 124 as shown in FIG. 2. In some embodiments, the outer wall 124 may have sharp edges. In some embodiments, the mask 102 may cover an entire face 106 of the patient 100. For example, the mask 102 may cover the eyes 107 and/or the forehead 122 of the patient.

FIG. 3 illustrates a cross section view of the patient 100 wearing the negative pressure procedure mask 102 according to an aspect of the present disclosure. The mask 102 may have an inner chamber 136. The inner chamber 136 may enclose the nose 108 and the mouth 110 of the patient 100. The inner chamber 136 may be fluidly connected to a negative pressure chamber 138. The negative pressure chamber 138 may open to the suction opening 134 (see FIG. 2) and the outer openings 126. Thus, the negative pressure chamber 138 may be walled by the seals 128 anteriorly and open to the inner chamber 136 posteriorly. A remote or external suction device coupled to the suction opening 134 may remove the aerosol particles located within the negative pressure chamber 138 and the inner chamber 136 while maintaining a slight negative pressure relative to rest of the mask 102 without causing discomfort to the patient 100. The negative pressure inside the negative pressure chamber 138 may prevent aerosol particles from exiting the mask 102. The negative pressure chamber 138 may be separated from the inner chamber 136 by an inner wall 140.

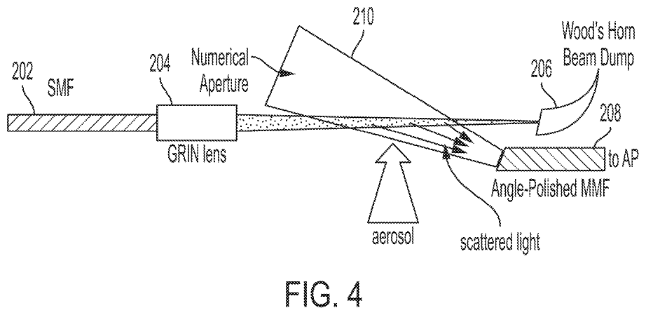

FIG. 4 illustrates an aerosol sensor 200 of the negative pressure procedure mask 102 (see FIG. 1) using light scattering to detect aerosol particles according to an aspect of the present disclosure. The mask 102 may have a first aerosol sensor located at or near the suction openings 134 (see FIG. 1). The first aerosol sensor may detect aerosol particles being removed from the suction device. The mask 102 may further have a second aerosol sensor located outside of the mask 102. The second aerosol sensor may detect aerosol particles escaping the mask 102, particularly the negative pressure chamber 138 (see FIG. 3). The aerosol sensor 200 may be a photoelectric sensor that uses light scattering to detect the presence of aerosols. The aerosol sensor 200 may resemble a sensor of a conventional smoke detector used in identifying the presence of smoke particulates in households. A coincidence detection technique similar to a logical detection technique may be used to avoid false positives. Both the first aerosol sensor and the second aerosol sensor being positive may indicate that the mask 102 has failed and an aerosol has been expelled outside the mask 102 into the examination room. In some embodiments, only the first aerosol sensor or the second aerosol sensor being positive may indicate that an aerosol has been contained.

Most scattered light is forward scattered, and thus a scattered light collecting single mode optical fiber (SMF) 202 of the aerosol sensor 200 may be placed near an optical axis. For example, a 488 nanometer (nm) diode laser may pass through the SMF 202. Also, other sizes of laser diodes are contemplated. The incoming laser from the SMF 202 may be refracted when it enters a gradient index (GRIN) lens 204. The laser may then pass the GRIN lens 204 until it exits the GRIN lens 204 where the laser is refracted again. A laser beam dump 206 may be used to absorb incident light and capture unwanted beams. A Wood's horn beam dump 206 is shown by example in FIG. 4. Other laser beam dumps 206 are contemplated, such as a cone dump. A multi-mode fiber (MMF) 208 may carry scattered light from the presence of any aerosol particles to an avalanche photodiode (AP). The numerical aperture of the scattered light is shown by the cone 210. The MMF 208 may have a fiber end face polished at an angle to prevent light that reflects from the interface from traveling back up the MMF 208. The AP may convert light into electricity to detect the scattered light, and thus detect the presence of any aerosol particles. The aerosol sensor 200 may provide visual, auditory, or haptic feedback when an aerosol particle is detected. For example, the aerosol sensor 200 may turn on a light, flash, change color, beep, sound an alarm, or vibrate.

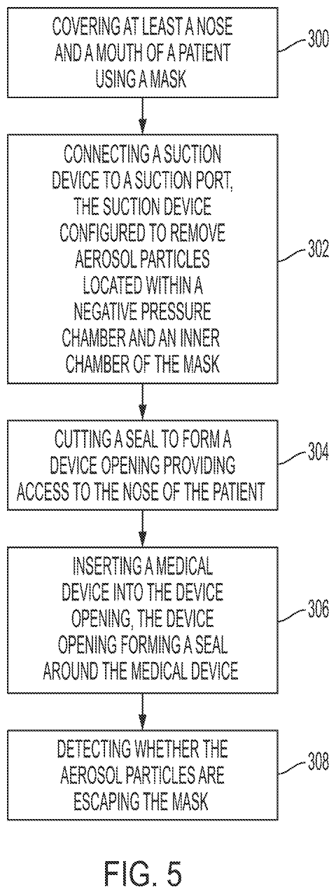

FIG. 5 is a flowchart illustrating a method for reducing exposure of aerosol particles emitted from a patient 100 (see FIG. 1) to other individuals according to an aspect of the present disclosure. The method may be performed with the mask 102 (see FIG. 1). The method may begin with block 300. In block 300, the method may include covering at least a nose 108 (see FIG. 3) and a mouth 110 (see FIG. 3) of the patient 100 using the mask 102. The mask 102 may be attached to the patient 100 by straps or loops 114 (see FIG. 1) that each go over an ear 116 (see FIG. 1) of the patient 100. Other ways to attach the mask 102 to the patient 100 are contemplated, such as arms that extend behind the ears 116 and securements around the neck 120 (see FIG. 1) or the forehead 122 (see FIG. 1) of the patient 100.

The method may continue with block 302. In block 302, the method may include connecting a suction device to a suction port or opening 134 (see FIG. 1). The suction device may be configured to remove aerosol particles located within a negative pressure chamber 138 (see FIG. 3) and an inner chamber 136 (see FIG. 3) of the mask 102.

The method may continue with block 304. In block 304, the method may include cutting or opening a seal 128 (see FIG. 1) to form a device opening 130 (see FIG. 1) providing access to the nose 108 of the patient 100. The device openings 130 may be configured to receive a medical device. For example, the medical device may be an endoscope or a laryngoscope. In some embodiments, pre-cut device openings 130 may be present on the seal 128 in lieu of this step. The device openings 130 may be shaped and sized to accommodate a wide range of patient anatomies.

The method may continue with block 306. In block 306, the method may include inserting the medical device into the device opening 130. The medical device may form a seal around the medical device. Once the medical device is inserted, the physician may perform a medical examination, procedure, or operation using the medical device without the patient emitting aerosol particles to the examination room.

The method may conclude with block 308. In block 308, the method may include detecting whether aerosol particles are escaping the mask 102. The mask 102 may have a first aerosol sensor located at the suction openings 134 (see FIG. 1). The first aerosol sensor may detect aerosol particles being removed from the suction device. The mask 102 may further have a second aerosol sensor located outside of the mask 102. The second aerosol sensor may detect aerosol particles escaping the mask 102, particularly the negative pressure chamber 138 (see FIG. 3). The aerosol sensor 200 may be a photoelectric sensor that uses light scattering to detect the presence of aerosols. A coincidence detection technique may be used to avoid false positives. Both the first aerosol sensor and the second aerosol sensor being positive may indicate that the mask 102 has failed and an aerosol has been expelled outside the mask 102 into the examination room. In some embodiments, only the first aerosol sensor or only the second aerosol sensor being positive may indicate that an aerosol has been contained.

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A mask for a patient for reducing exposure of aerosol particles emitted from the patient to other individuals, the mask comprising:
an inner chamber configured to enclose a nose and a mouth of the patient; and
a negative pressure chamber fluidly connected to the inner chamber and having an outer opening and a suction opening, the outer opening covered by a seal and the suction opening configured to couple to a suction device for removing the aerosol particles located within the inner chamber and the negative pressure chamber.

2. The mask of claim 1, wherein the seal is configured to be cut or opened to form a device opening providing access to the nose of the patient, the device opening receiving a medical device and forming a seal around the medical device.

3. The mask of claim 2, wherein the medical device is an endoscope.

4. The mask of claim 1, further comprising an outer wall defining:
a posterior opening for surrounding the nose and the mouth of the patient,
the suction opening of the negative pressure chamber,
the outer opening of the negative pressure chamber, and
a filter opening configured to receive and house a filter for filtering air moving between the inner chamber and outside of the mask.

5. The mask of claim 4, further comprising an inner wall defining the negative pressure chamber.

6. The mask of claim 1, further comprising a first aerosol sensor located at the suction opening and configured to detect the aerosol particles being removed by the suction device.

7. The mask of claim 6, further comprising a second aerosol sensor located outside of the mask and configured to detect the aerosol particles escaping the negative pressure chamber.

8. The mask of claim 7, wherein the first aerosol sensor and the second aerosol sensor use light scattering to detect the aerosol particles.

9. A mask for a patient to be worn during a medical examination to mitigate exposure of aerosol particles emitted from the patient into an examination room, the mask comprising:
a negative pressure chamber having an outer opening and a suction opening, the suction opening configured to couple to a suction device for removing the aerosol particles located within the negative pressure chamber; and
a seal covering the outer opening having a device opening providing access to a nose of the patient, the device opening receiving a medical device and forming a seal around the medical device.

10. The mask of claim 9, wherein the device opening is cut with a sharp instrument prior to receiving the medical device.

11. The mask of claim 9, further comprising an inner chamber fluidly connected to the negative pressure chamber and configured to enclose the nose and a mouth of the patient.

12. The mask of claim 9, wherein the medical device is an endoscope.

13. The mask of claim 9, further comprising an outer wall defining:
a posterior opening for surrounding the nose and the mouth of the patient,
the suction opening of the negative pressure chamber,
the outer opening of the negative pressure chamber, and
a filter opening configured to receive and house a filter for filtering air moving between the inner chamber and outside of the mask.

14. The mask of claim 13, further comprising an inner wall defining the negative pressure chamber.

15. The mask of claim 9, further comprising a first aerosol sensor located at the suction opening and configured to detect the aerosol particles being removed by the suction device.

16. The mask of claim 15, further comprising a second aerosol sensor located outside of the mask and configured to detect the aerosol particles escaping the negative pressure chamber.

17. The mask of claim 16, wherein the first aerosol sensor and the second aerosol sensor use light scattering to detect the aerosol particles.

18. The mask of claim 17, wherein the first aerosol sensor and the second aerosol sensor simultaneously detecting the aerosol particles indicate that the aerosol particles have escaped into the examination room.

19. A method for reducing exposure of aerosol particles emitted from a patient to other individuals, the method comprising:
covering at least a nose and a mouth of the patient using a mask, the mask having:
an inner chamber configured to enclose the nose and the mouth of the patient, and
a negative pressure chamber fluidly connected to the inner chamber and having an outer opening and a suction opening, the outer opening covered by a seal;
connecting a suction device to the suction port, the suction device configured to remove the aerosol particles located within the negative pressure chamber and the inner chamber;
cutting or opening the seal to form a device opening providing access to the nose of the patient; and
inserting a medical device into the device opening, the device opening forming a seal around the medical device.

20. The method of claim 19, further comprising detecting whether the aerosol particles are escaping the mask using a first aerosol sensor located at the suction opening and configured to detect the aerosol particles being removed by the suction device and a second aerosol sensor located outside of the mask and configured to detect the aerosol particles escaping the negative pressure chamber.
